(19) United States
(12) Patent Application Publication
(10) Pub. No.: US 2025/0241793 A1
(43) Pub. Date: Jul. 31, 2025

(54) MODIFICATION OF TISSUE PROPERTIES VIA PHOTOMODULATION

(71) Applicant: University of Pittsburgh - Of the Commonwealth System of Higher Education, Pittsburgh, PA (US)

(72) Inventors: Ian Alejandro Sigal, Pittsburgh, PA (US); Po-Yi Lee, Belmont, MA (US)

(21) Appl. No.: 19/037,151

(22) Filed: Jan. 25, 2025

Related U.S. Application Data
(60) Provisional application No. 63/625,399, filed on Jan. 26, 2024.

(51) Int. Cl. A61F 9/008

(52) U.S. Cl. CPC A61F 9/008

(57) ABSTRACT

A method of modifying biomechanical properties of tissue in a region of interest includes application of laser energy to one or more first volumes of tissue in the region of interest to cause softening via photodegradation, and application of laser energy to one or more second volumes of tissue within the region of interest to cause stiffening via photocrosslinking. The one or more second volumes are different from the one or more first volumes.

### CROSS-REFERENCE TO RELATED APPLICATIONS

This application claims benefit of U.S. Provisional Patent Application Ser. No. 63/625,399, filed Jan. 26, 2024, the disclosure of which is incorporated herein by reference.

### GOVERNMENTAL INTEREST

This invention was made with government support under grant number EY023966, awarded by the National Institutes of Health. The government has certain rights in the invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosures of all references cited herein are incorporated by reference.

Glaucoma is a group of eye diseases that results in vision loss and even blindness. An estimated 76 million people have glaucoma, a number expected to increase to 112 million in 2040. Primary open-angle glaucoma, the most common type of glaucoma, develops slowly and usually has no symptoms. Vision loss in glaucoma involves not only progressively narrowing the visual field, but also worsening the quality of vision. Glaucomatous vision loss is irreversible. If left untreated, glaucoma can eventually result in blindness.

Blindness in glaucoma is due to the loss of retinal ganglion cell axons that carry visual information to the brain. The irreversible loss of the retinal ganglion cell axons initiates in the optic nerve head (ONH). The neurodegeneration initiates within the lamina cribrosa (LC), which is surrounded by the peripapillary sclera (PPS). The lamina cribrosa or LC (see FIG. 1) is a trabecular structure including an intricate network of collagenous beams (formed from collagen fibers) and blood vessels that support the delicate retinal ganglion cell (RGC) axons passing through the pores.

FIG. 1 is a coronal view image of the eye. The central part of the image shows the LC, with collagenous beams radiating from the central retinal artery and vein. Circumferential fibers form the closest ring around the canal. Further out are tangential fibers that carry stresses away from the canal to the peripheral sclera. A complex network of interwoven fibers is visible near the edge of the image.

Elevated intraocular pressure (IOP) is a major risk factor for the development and progression of glaucoma. A primary theory of axon loss is IOP-induced mechanical insult to the axons, including, for example, compression, stretch, and shear. Specifically, IOP-induced scleral deformations are transmitted to the scleral canal, leading to excessive biomechanical strains on LC and then damage to retinal ganglion cell axons within the LC pores. Hence, all current clinically approved treatments aim to lower IOP. Unfortunately, 25% to 45% of these patients still eventually lose vision despite a decrease in their IOP. Other patients have vision loss at statistically normal IOP. In contrast, some have slow progression and low impact on their vision at even high IOP. Such outcomes indicate people have different sensitivities to IOP.

From a biomechanical standpoint, variations in the ability of the eyes to provide mechanical support to the axons may explain the sensitivity of a specific eye to IOP and its susceptibility to glaucomatous axonal damage. Some eyes may provide better mechanical support, reducing IOP-induced neural tissue insult to the axons and protecting them from damage. Conversely, other eyes may be particularly frail, increasing insult to the axons and contributing to the cascade of events that eventually lead to vision loss, potentially even at an otherwise normal intraocular pressure. Following such a biomechanical standpoint, biomechanical markers may be identifiable that indicate the level of susceptibility to glaucoma and sensitivity to IOP. Once such markers are identified, preventative screenings will help identify high-risk groups for glaucomatous vision loss. Furthermore, novel therapeutic approaches are required to treat glaucoma based on altering the mechanical properties of the eye to reduce the mechanical insult to axons.

Mechanisms have been proposed to reduce the mechanical insult to the axons, usually through stiffening the tissues of the back of the eye, specifically the sclera. In that regard, it was hypothesized that stiffening the sclera to reduce IOP-induced scleral deformations may prevent LC from excessive biomechanical strains and thus protect against the loss of retinal ganglion cells in glaucoma. Studies of scleral stiffening in the treatment of glaucoma have, however, shown mixed results. Methods for stiffening using viral vectors and chemical agents have been proposed, but they all have significant complications including, but not limited to, invasiveness, toxicity, and/or difficulties in spatial control.

To achieve stiffening via inducing collagen crosslinking, studies or clinical practices have applied chemical agents to connective tissues. These chemical agents are categorized into photosensitized and non-photosensitized. Photosensitized agents are based on electronic transitions from photon absorption by photosensitizers, where excited photosensitizers react with the protein and thereby initiate collagen crosslinking or degradation. For example, riboflavin is a clinically-approved photosensitized crosslinking for keratoconus, a corneal disorder. The treatment is based on riboflavin photoactivated by ultraviolet-A (UVA) light to induce an increase in corneal stiffness. That approach allowed targeting riboflavin photocrosslinking, achieving selective stiffening. To improve biocompatibility and treatment resolution, another study replaced UVA light with near-infrared femtosecond laser as a light source to produce spatially controlled collagen crosslinking and mechanical stiffening within the cornea. Other photosensitizers, such as methylene blue, have been proposed for in vivo scleral stiffening. However, using a photosensitizer may cause photodamage after the irradiation, which might induce further complications after healing. In contrast to the need for light activation for photosensitized crosslinkers, non-photosensitized crosslinkers are based on molecule diffusion through tissues to directly react with collagen on the way. Glyceraldehyde, genipin, and methylglyoxal are primary non-photosensitized crosslinkers. Since the diffusion process is quick and unbounded, non-photosensitized agents lack selectivity on regional stiffening or softening. Most importantly, both non-photosensitizer and photosensitizer crosslinkers are limited by drug delivery to the deep tissue.

Collagenase (an enzyme that breaks down collagen) has been used to change the biomechanics in the eye (via inducing collagen degradation). For example, collagenase can break down excessive crosslinks in the cornea and improve the corneal shape, and thus has been proposed as a potential treatment for corneal ectasia, a condition in which the cornea thins and bulges outwards, causing distorted vision. However, as a result of a lack of selectivity, collagenase may further weaken the collagenous stroma, resulting in deformations of the tissue structure.

Laser ablation is a standard method to change collagen thickness for eye surgery, such as laser-assisted in situ keratomileusis (LASIK) and photorefractive keratectomy (PRK). Basically, laser ablation is based on a high-energy, focused laser to heat and remove the local tissue. Gas bubbles are routinely formed since the collagen change is based on thermal or thermoacoustic effects. The gas bubble formation during eye surgery carries potential risks and complications, such as increased intraocular pressure, infection, and corneal damage.

### SUMMARY

A method of modifying biomechanical properties of tissue in a region of interest includes application of laser energy to one or more first volumes of tissue in the region of interest to cause softening via photodegradation, and application of laser energy to one or more second volumes of tissue within the region of interest to cause stiffening via photocrosslinking. The one or more second volumes are different from (or spaced from) the one or more first volumes. In a number of embodiments, the method further includes determining modifications of the biomechanical properties of the tissue in the region of interest and determining the one or more first volumes of tissue and the one or more second volumes of tissue to achieve the determined modifications prior to application of laser energy to the one or more first volumes of tissue and application of laser energy to one or more second volumes of tissue. The method may, for example, further include creating a biomechanical index or map of the region of interest before application of laser energy to the one or more first volumes of tissue and application of laser energy to the one or more second volumes of tissue.

Each of the one or more first volumes of tissue and the one or more second volumes of tissue may include collagen. In a number of embodiments, a first laser is used to apply laser energy to the one or more first volumes of tissue and a second laser is used to apply laser energy to the one or more second volumes of tissue. In other embodiments, a single laser is used to apply laser energy to the one or more first volumes of tissue and to the one or more second volumes of the tissue.

The biomechanical properties of the tissue are modified through (photoinduced) thermoacoustic effects or through photochemical effects. In a number of embodiments, the biomechanical properties of the tissue are modified in the absence of thermoacoustic effects.

In a number of embodiments, a femtosecond laser is used for at least one of application of laser energy to the one or more first volumes of tissue and for application of laser energy to the one or more second volumes of tissue. A femtosecond laser may be used for application of laser energy to the one or more first volumes of tissue and for application of laser energy to the one or more second volumes tissue. The total time of application of laser energy to each of the one or more first volumes of tissue may, for example, be less than the total time of application of laser energy to each of the one or more second volumes of tissue.

In a number of embodiments, each of the one or more first volumes of tissue and each of the one or more second volumes of tissue is tissue of the eye. Each of the one or more first volumes of tissue and each of the one or more second volumes of tissue is tissue of the trabecular meshwork, Schlemm's canal, the episclera, the sclera or tissue of the lamina cribrosa. In a number of embodiments, each of the one or more first volumes of tissue and each of the one or more second volumes of tissue is tissue of the lamina cribrosa. In a number of embodiments, each of the one or more first volumes of tissue and each of the one or more second volumes of tissue are located on one or more beams of the lamina cribrosa which do not include a blood vessel or in a volume of a beam through which a blood vessel does not pass. The biomechanical properties of the tissue may, for example, be modified to treat glaucoma or to prevent glaucoma.

The laser energy applied to the one or more first volumes of tissue and the laser energy applied to the one or more second volumes of tissue may, for example, have an axial resolution of not more than 40 μm, not more than 30 μm, not more than 20 μm, or not more than 10 μm. The laser energy applied to the one or more first volumes of tissue and the laser energy applied to the one or more second volumes of tissue may, for example, have a lateral resolution of not more than 40 μm, not more than 30 μm, not more than 20 μm, or not more than 10 μm.

In a number of embodiments, the region of interest is tissue of the eye, and creating the biomechanic index includes comparison of images created at different pressures within the eye. The different pressures may be created either intrinsically or extrinsically. As used herein, intrinsically created pressure differences are pressure differences arising from a body function such as the heartbeat. Extrinsically created pressure differences are pressure differences created by externally applied means such as a force applied to the eye.

A method of treating or preventing glaucoma includes softening tissue in a region of interest of the eye. The tissue may be collagenous tissue. The collagenous tissue of the eye may, for example, be collagenous tissue of at least one of the trabecular meshwork, Schlemm's canal, the episclera, the sclera and the lamina cribrosa. In a number of embodiments, the collagenous tissue of the eye is collagenous tissue of the sclera or the lamina cribrosa. In a number of embodiments, the collagenous tissue of the eye is collagenous tissue of the lamina cribrosa. In a number of embodiments, the tissue in the region of interest is softened via the application of laser energy. The method may, for example, further include applying laser energy to one or more second volumes of collagenous tissue of the eye to cause stiffening in the region of interest. The one or more second volumes are different from the one or more first volumes.

A method of modifying biomechanical properties of tissue in a region of interest includes application softening tissue in one or more first volumes of tissue in the region of interest and stiffening one or more second volumes of tissue within the region of interest. The one or more second volumes are different from the one or more first volumes.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1 is an image illustrating a coronal view of the eye.

FIG. 2A illustrates a stress-strain curve demonstrating that the stiffness of the tissues can be simplified as the slope of a stress-strain curve.

FIG. 2B illustrates the effects of chemical reactions on collagen wherein the stiffness of the tissues is increased via collagen crosslinking, which strengthens chemical bonds between collagen fibrils and thus increases structural stiffness.

FIG. 2C illustrates the effects of chemical reactions on collagen stiffness wherein collagen degradation weakens tissues and thus decreases structural stiffness.

FIG. 3 illustrates an embodiment of a portion of system hereof for application of laser energy to modify the biomechanical properties of collagenous tissue.

FIG. 4 illustrates a representative example of a process hereof including preoperative screening and biomechanics indexing to perform a determination of volumes of tissue for stiffening via photocrosslinking and volumes of tissue for softening via photodegradation/photoscalpel, and post treatment results.

FIG. 5 illustrates a schematic representation of the process of FIG. 4.

FIG. 6 illustrates photomicrographs setting forth the results of repeated photomodulation treatments on collagen, wherein multiphoton images (darker gray: second harmonic generation (SHG) microscopy; lighter gray to white: two-photon excitation autofluorescence (TPAF) microscopy) of sheep cornea show the process of collagen photomodulation during repeated treatments.

FIG. 7 illustrates resolution testing of photomodulation, wherein multiphoton images (darker gray: SHG; lighter gray: TPAF) and signal profiles of SHG and AF before and after linear photomodulation on sheep lamina cribrosa beams.

FIG. 8A is an image illustrating photoscalpel visualization using instant polarized light microscopy (IPOL) on a sheep lamina cribrosa beam using multiphoton microscopy (SHG, TPAF)

FIG. 8B is an image illustrating IPOL to set forth a brightness profile along the dash line on the right side of the figure.

FIG. 8C illustrates brightness as a function of distance along the dashed line on the right side of FIG. 8B, wherein the image of FIG. 8B and the graph of FIG. 8C provide evidence of collagen loss instead of photobleaching after photomodulation.

FIG. 9A is an image of a sheep lamina cribrosa beam having undergone photoscalpel treatment compared to the untreated region wherein the loss of SHG signals after photomodulation indicates the occurrence of photoscalpel collagen degradation.

FIG. 9B is an image illustrating that bending collagen fiber bundles were observed near the photomodulation region (white rectangle), wherein the bundles were straightened after stretching (represented by white arrows).

FIG. 9C is an image illustrating that regions with a higher strain (arrows) were located at the treated region, indicating a decrease in stiffness.

FIG. 10A is an image illustrating that there was no significant change in SHG and TPAF signals after photomodulation (photocrosslinking).

FIG. 10B is an image illustrating the strain of the lamina cribrosa beam with photocrosslinking treatments which was measured by stretch testing.

FIG. 10C is an image illustrating a region with a lower strain (arrow) located at the treated region, which indicates an increase in stiffness and the occurrence of photocrosslinking.

FIG. 11A illustrates schematically a proposed theory of the dominant chemical reaction of photomodulation wherein free electrons induced by a femtosecond laser were viewed as a chemical reactant, and wherein, when the concentration of free electrons is low, collagen crosslinking and the formation of photoproducts are the dominant chemical reaction.

FIG. 11B illustrates schematically a proposed theory of the dominant chemical reaction of photomodulation wherein free electrons induced by a femtosecond laser were viewed as a chemical reactant, and wherein, when the concentration of free electrons is high, collagen degradation is the dominant chemical reaction and thus causes collagen loss.

FIG. 12 illustrates a representative embodiment of system hereof for application of laser energy of tissues of the eye in vivo.

FIG. 13 illustrates schematically and via a photographic image an embodiment of an IPOL microscope used in studies hereof (for example, in visualizing bubbles and in determining collagen orientation).

FIG. 14 illustrates schematically that strain difference between treated and untreated regions determines photomodulation effects, wherein the optic nerve head section is imaged and subjected to biaxial and quasi-static stretching, and a local lamina beam approximates a linear, uniform, and simple structure, and allows for the stretch to be observed as uniaxial elongation, and wherein, when the displacement at the boundaries of the beam is assigned, softer regions undergo larger deformation compared to stiffer regions, and further wherein: (A) lower strain in the treated region indicates an increased stiffness due to photomodulation treatment, and (B) a higher strain in the treated region indicates a decreased stiffness due to photomodulation treatment.

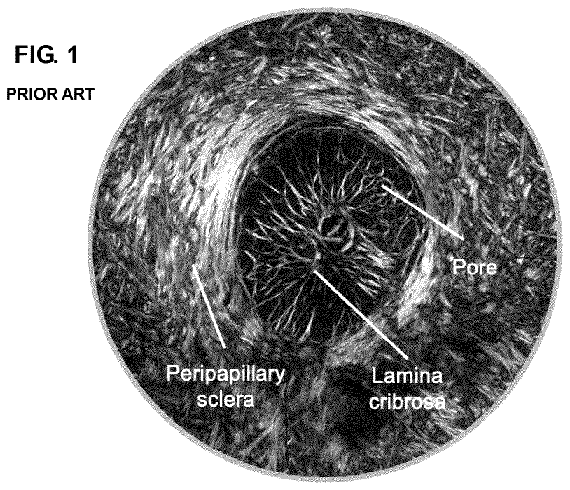

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “an”, and “the” include plural references unless the context clearly dictates otherwise. Thus, for example, reference to “a volume” includes a plurality of such volumes and equivalents thereof known to those skilled in the art, and so forth, and reference to “the volume” is a reference to one or more such volumes and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry,” “circuitry” or “circuit,” as used herein include, but are not limited to, hardware, firmware, software, or combinations of each to perform a function(s) or an action(s). For example, based on a desired feature or need, a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic,” as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random-access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separately from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

As used herein, the term “femtosecond laser” refers to a laser which can focus energy on a short time scale within a single laser pulse. The short or ultrashort pulses of light may, for example, have a duration in the range of approximately 10-15 to approximately 10-12 seconds (that is, in the range of femtoseconds).

Various aspects of embodiments of devices, systems, and methods hereof are discussed herein in connection with at least one of stiffening and softening of tissue of the eye in, for example, the treatment or prevention of glaucoma. However, one skilled in the art will appreciate that the devices, systems, and methods hereof may be used in connection with procedures (which may be at least one of therapeutic or diagnostic) other than treatments associated with glaucoma and in connection with tissue(s) other than tissue(s) of the eye.

As described above, glaucomatous vision loss starts with damage to the retinal ganglion cell axons, which exit the globe through the lamina cribrosa or LC, a structure within ONH in the posterior pole of the eye. The causes for the axonal damage at the LC have not been completely identified, although significant evidence points to mechanical insult as a primary damage trigger. Determining how the LC resists mechanical insult remains a significant challenge, largely as a result to the lack of imaging techniques capable of visualizing the LC with high-resolution, wide field-of-view, and high-speed, as well as the associated experimental techniques that allow evaluating LC biomechanics and the effects of load. In a number of embodiments hereof, novel approaches for imaging and experimental testing are described which enable a better understanding of the ONH architecture and biomechanics. Further devices, systems, and methods for altering biomechanics of tissue, including tissue of the eye are described.

In a number of embodiments hereof, improved polarized light imaging methods were leveraged to characterize ONH collagen architecture and biomechanics, and techniques were to developed to modify the mechanical properties of ONH collagen, and visualizing and analyzing the ONH vasculature. In that regard, polarized light imaging techniques were developed to visualize and quantify collagen microstructure and orientation in real-time, without labeling or stains. These techniques were integrated with a customized micro-stretcher to characterize the deformations and nonlinear mechanical behavior of the ONH. Further, laser techniques (for example, femtosecond laser techniques) were introduced to modify the mechanical properties of the LC with micrometer precision, without the need for photosensitizers. By varying the laser parameters, it is possible to induce one or both of collagen crosslinking to stiffen tissues and collagen degradation to soften tissues.

Once again, significant evidence suggests that damage to retinal ganglion cells in glaucoma occurs within LC, where significant physical tissue deformation occurs. From a biomechanical standpoint, reducing LC deformations within the ONH is a way to increase the resistance to such mechanical insults. Some studies have demonstrated that high scleral stiffness reduces mechanical strain in the LC at elevated IOP. It has been hypothesized that stiffening the sclera to reduce IOP-induced scleral deformations may prevent LC from excessive biomechanical strains and thus protect against the loss of retinal ganglion cells in glaucoma. Following this theory, multiple collagen crosslinking approaches (for example, non-photosensitizer and photosensitizer chemical agents) for sclera have been reported the effectiveness in scleral stiffening. Collagen crosslinking refers to the ability of collagen fibrils to form strong chemical bonds with adjacent fibrils, and thus can be used to stiffen soft tissues, such as cornea and sclera. Although several studies aiming for scleral stiffening have shown some progress, studies of a mice model revealed contradictory results. Not every eye benefits from scleral stiffening. There are a number of explanations for this opposite result. First, the stiffening method may have lacked spatial selectivity and thus stiffened the whole globe, which may increase IOP spikes and thus damage the retinal ganglion cell axons. Therefore, controlling the extent of the treatment zone may highly affect the treatment result. Second, over-stiffening may have detrimental effects on the vascular or biochemical response of the ONH. The effect of collagen crosslinking on tissue stiffness may vary between individuals. Third, collagen crosslinking agents may cause issues of biocompatibility. Those explanations indicate the need for developing improved approaches to modulating ocular biomechanics to treat glaucoma.

A computational study of LC biomechanics has indicated that there was a significant correlation between the geometric parameters of an LC pore and the level of biomechanical insult to the neural tissues within the pore. A follow-up study found that missing LC beams may be able to mitigate TOP-induced neural tissue insult, suggesting that the role of the LC connective tissues is more complex than simply fortifying against IOP. This result suggested that removing LC beams can mitigate IOP-induced neural tissue insult, which opens the possibility that biomechanical modulation on LC can be used for glaucoma treatment. However, there is no suitable approach to remove the collagen beams, and thus no experimental data has been reported to verify the theory. Collagenases have been used to achieve a decrease in stiffness of the sclera and LC. However, such an approach lacks spatial selectivity and may damage the axon within the LC. In addition, collagenases may weaken the collagenous stroma and thus lead to deformations of the tissue structure.

Although, tissue biomechanical modulation has been proposed as a potential therapeutic approach to manage glaucomatous optic neuropathy, inconsistent preclinical data indicates a need for innovative approaches to alter ONH biomechanics. In addition, many aspects of biomechanical modulation still have not been characterized. The susceptibility of ONH biomechanics to glaucoma as well as the feasibility of modulating ONH biomechanics as a potential treatment for glaucoma require further study. It is, for example, important to more fully understand differences in tissue behavior arising from between the original and changes in ONH microarchitecture and overall morphology. Such observations demonstrate a need for innovative tools and approaches to characterize the local mechanical properties of ocular tissues.

Ocular biomechanics has been associated with natural processes like development and aging and with several eye disorders such as myopia, keratoconus, and glaucoma. Several approaches have been deployed to characterize the biomechanical properties of posterior pole tissues ex vivo, including tensile/compression testing, whole globe inflation testing, etc. In most studies, such approaches aim to build stress-strain curves or pressure-strain curves. For instance, whole globe inflation testing allows calculating localized circumferential strains and correlating IOP with multiaxial regional deformation. Although that technique presents minimal boundary conditions and near physiologic loading and can identify the regional difference in biomechanics, there is no information on why these regions have different mechanical properties. Collagen is the major load-bearing component of the eye and thus plays a central role in determining tissue biomechanics. Collagen is fibrous in the posterior pole, and the architecture highly affects the biomechanics of the eye. Collagen fibers and fiber bundles form complex networks in the eye. On the mesoscale level, collagen architecture (such as density, anisotropy, and interweaving) affects stiffness. On the microstructural level, the waviness of the collagen fibers, or crimp, affects the nonlinear mechanical properties. How the collagen architecture of an eye determines its mechanical behavior is an area of significant interest in ocular biomechanics.

Image-based stretch testing is a powerful and direct way to characterize the change in collagen architecture under stretch. Many imaging techniques have been used for measuring and/or visualizing the architecture and biomechanics of collagen in the eye. These include confocal, nonlinear, microscopies, and optical coherence tomography. Each of those techniques offers a unique combination of resolution, field of view, penetration depth, speed, and tissue specificity. Such techniques, although useful, have one or more limitations such as a small field of view, low resolution, subjective and slow analysis, introduction of artifacts, and high expense, which translates into restricted availability.

Over the past several years, polarized light microscopy (PLM) has been demonstrated to allow visualization and quantification of ONH collagen tissues with micrometer-scale resolution over wide regions. A variation of PLM, a combination of a circular polarizer and a linear polarizer, has proven useful for the study of collagenous tissues ex vivo, revealing patterns of fiber architecture throughout the globes of humans and other animals. The high angular resolution of PLM allows measurement of the degree of stretch or relaxation of collagen fibers, also referred to as crimp, which eludes other imaging techniques. Conventional PLM, however, requires multiple images acquired under various polarization states (four in various implementations hereof), which slows down imaging speed. Hence, previous studies using PLM were limited to static evaluation of tissues from eyes fixed at different IOPs. That methodology is particularly limiting for the study of the mechanical behaviors of sclera and LC beams. Even though multiple channel acquisition can overcome the issue of imaging speed, PLM still requires post-processing, such as image registration and noise reduction. Such post-processing decreases resolution and may lose important details of the ocular sections. In a number of embodiments hereof, a novel PLM techniques desirably allows visualizing and quantifying collagen microstructure and orientation in a single color image without any post-processing. In that regard, instant polarized light microscopy or IPOL encodes optically information about fiber orientation and retardance through a color snapshot. The technique is compatible with stretch testing, and thus can be used to characterize stretch-induced deformation of collagen microstructures of ONH tissues.

As described above with respect to modification of eye tissue in treating glaucoma, although some studies have demonstrated that scleral stiffening is a possible neuroprotective therapy in glaucoma, the results are mixed. For example, an in vivo study reported that whole-globe scleral stiffening increased glaucoma damage in a mouse model. One explanation for this negative result is that ubiquitous alterations in corneoscleral stiffness caused larger IOP spikes and thus increased the glaucoma risk. Another explanation for the observed result is that over-stiffening may alter the vascular and/or biochemical response of the ONH. Such observations points to a need for a more rational approach that can alter tissues more precisely than the hammer of stiffening everything. In some cases, some degree of tissue compliance is beneficial to avoid concentrations of stress or localized deformations that could trigger damage. That may indicated a need for a tissue-softening approach that can be used to balance the stiffening effects on the ONH to avoid worsening. Although a number of methodologies, including chemical-agent-based techniques may be used to modulate biomechanical properties of tissue (that is, increase or decrease stiffness thereof via collagen crosslinking or degradation, respectively), such chemical techniques have significant associated drawbacks as discussed above.

The application of energy such as light/laser energy is desirable in the devices, systems, and methods hereof to provide significant improvements for modulating collagen properties of the ONH in high resolution. For example, a femtosecond laser approach enables selective crosslink formation without using photosensitized crosslinkers. Based on the histological and confocal imaging results, non-ablative, femtosecond laser-induced crosslinking does not cause thermal or thermomechanical damage. It has been further demonstrated that a femtosecond laser approach enables engraving 3D spatial features with the precision of less than 2 μm in soft tissues without using any photosensitized agents and without thermal or thermomechanical effects. Based on the integration of such approaches, modulating the mechanical properties of the LC to reduce the mechanical insult to axons via femtosecond laser is used in a number of embodiments hereof as a therapeutic paradigm for glaucoma.

In addition to the mechanical factors, it has long been believed that glaucomatous axon damage could also result from the vascular factor, an insufficient oxygen supply within the ONH resulting from compromised blood flow. The blood vessels of the ONH form a complex network intertwined with the collagen beams in the lamina cribrosa. The close relationship between the collagenous and vascular networks within the LC suggests that these two factors likely have substantial effects on one another. When modifying collagen architecture to release the mechanical insult, it is desirable to avoid directly damaging vessels as well as any resulting changes in mechanical support to vessels should be considered. On one hand, it is possible to target beams that do not contain a vessel. In that regard, the present inventors recently discovered that over half of lamina cribrosa collagen beams do not contain a blood vessel instead of a one-to-one relationship between collagen beams and vessels. Waxman, S., et al., Lamina cribrosa vessel and collagen beam networks are distinct. Experimental eye research, 215, 108916 (2022). On the other hand, collagen beams of the LC provide mechanical support to local microvasculature, and thus blood perfusion may be sensitive to changes in intraocular pressure or changes in collagen biomechanics. Vessels outside beams, however, may not be equally protected mechanically by the collagen and thus may be more sensitive to biomechanical changes. In addition, collagen beams that contain a vessel may be mechanically different from beams that do not have an opening for a vessel. Overall, it is important to understand the mechanical and spatial relationships between the collagenous and vascular networks of the LC to understand and consider their roles in susceptibility to modifying collagen architecture and glaucoma before modification.

The above discussion indicates a need for visualizing and characterizing the vascular network in the ONH. The vessels of the ONH can be fairly small-10 to 20 μm in diameter, and deep-several hundred micrometers from the optic disk surface. In addition, some of the vessels are enclosed within collagen beams. Current tools for visualizing posterior pole vasculature in vivo do not have sufficient resolution or imaging depth for certain in vitro characterization studies hereof. For example, optical coherence tomography (OCT) angiography has a high spatial resolution and provides excellent data on the retina and in some regions of the LC, making it suitable for in vivo applications hereof. However, it does not have sufficient imaging depth to visualize the vessels deep inside the ONH for a number of in vitro studies hereof. Ultrasound and magnetic resonance imaging have a high imaging depth but do not have the spatial resolution necessary to discern the small vessels of the ONH. Because of the importance of characterizing the ONH vasculature, there have been many attempts to do that ex vivo. One of the most successful was the use of vascular castings, often made in plastic. Analysis of the vascular casts, however, required destroying the rest of the tissues using corrosion methods, which precludes mechanical testing and precisely identifying the location of vessels relative to known non-vessel components, such as collagen. In contrast to the limitations of vascular casts, histological imaging with fluorescence labeling remains a powerful alternative to visualize the vessels of the ONH. Histological imaging allows for high spatial resolution imaging and characterization of the spatial relationship between vessels and collagen. The depth of study is a matter only of studying enough sections.

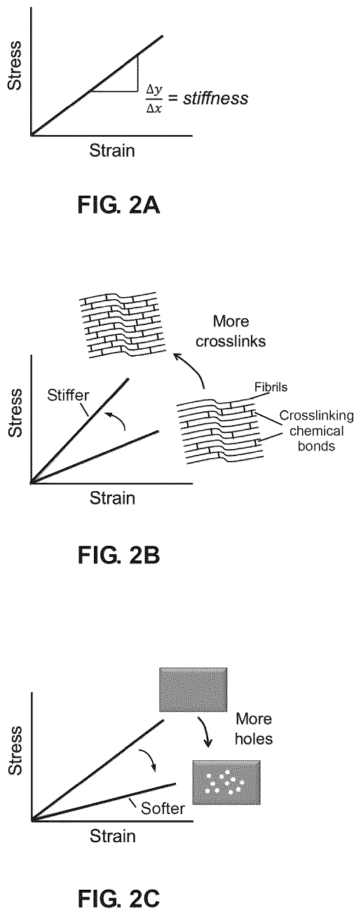

Once again, collagen is the primary structural component of connective tissues in the human body, such as skin and eye. Collagen properties determine tissue biomechanics, wherein the term “biomechanics” refers to how biological tissues respond under strain or stress (see, for example, FIGS. 2A through 2C). The strength of the chemical bond connection among collagen fibrils or molecules is one of the key factors in the mechanical properties and structural stability of collagen tissues and collagen-based biomaterials. Collagen crosslinking and degradation allow strengthening and weakening collagen chemical bonds, respectively, and have been associated with changes in tissue stiffness. Specifically, collagen crosslinking increases structural stiffness (FIG. 2B), whereas collagen degradation decreases structural stiffness (FIG. 2C).

Femtosecond laser irradiation may be used without using a photosensitizer to induce either collagen crosslinking or collagen degradation. The absorption of multiple photons may, for example, result in the photoionization of water and organic molecules, and subsequent chemical reactions became possible in the submicron focal region. Without limitation to any mechanism, femtosecond laser irradiation with suitable energy is capable of generating free electrons at sufficiently high density and then inducing photochemical reactions among collagen fibers. The photochemical reactions can be either collagen crosslinking or collagen degradation. The ability to control both photochemical reactions, which is sometimes referenced herein as photomodulation, may thus have applications in tissue engineering and therapeutic procedures. However, because gaps remain in the understanding of the effects of femtosecond laser on collagenous fibers and tissues, it is important to understand and characterize the photomodulation mechanism of collagen under femtosecond laser irradiation.

A number of studies hereof were carried out to improve the understanding of photomodulation treatments. Studies hereof included, for example, characterizing femtosecond laser effects on collagen, including building the relationship between photocrosslinking and photoscalpel processes, measuring spatial resolution, identifying structural change after photomodulation, and characterizing the change in biomechanics induced by photomodulation.

In a number of embodiments, devices, systems, and methods hereof modulate the biomechanics of the tissues via, for example, photomodulation as set forth above. Such photomodulation can be controlled to produce at least one of tissue stiffening or tissue softening. Tissue stiffening and softening can be used in a procedure with spatial separation to achieve a desired biomechanical modulation of the tissue. In a number of representative embodiments, photomodulation of the biomechanics of the tissues in the eye (for example, the back of the eye) is performed with the aim of improving the resilience of the eye to prevent or slow down pathology. For example, laser irradiation may be used to induce changes in collagenous tissue that, in turn, affect the tissue mechanics. In that regard, collagen stiffening can, for example, be induced by using short-duration, low-energy laser radiation (for example with a femtosecond laser) that induces collagen crosslinking. Softening can be induced by longer-duration laser irradiation (relative to the stiffening procedure) to induce degradation. The mechanisms for photomodulation can be achieved (without the use of photosensitizers or photosensitizing chemicals) through thermoacoustic effects (either high-energy laser or non-femtosecond laser) or through photochemical effects (low-energy femtosecond laser in the absence of thermoacoustic effects). In decreasing stiffness, either method works by causing collagen degradation and locally decreased stiffness (increased compliance). A combination of short-term and long-term irradiation can, for example, be used to modulate the mechanical properties of ocular (for example, LC) tissues, stiffening some regions and softening others in a predetermined manner, to route the forces and distortions that eventually trigger the cascade of events that cause vision loss in a predetermined manner. Such a rational method represents a new strategy for eye care (for example, to treat and/or prevent glaucoma). Moreover, such modulation of mechanical properties can be achieved in tissues other than ocular tissues to achieve various results or treat various conditions.

As described above, biomechanical modulation strategies currently under study at the back of the eye to treat and prevent glaucoma are based solely on tissue stiffening, and, particularly, stiffening of the sclera. However, stiffening is only one avenue to protect such tissues. Once again, while several studies aiming for tissue stiffening have shown some positive results in preventing glaucoma in animal models, such results are fairly limited. Moreover, results indicate that not every eye benefits from stiffening. Studies of the present inventors of ocular biomechanics indicate that some eyes will potentially benefit the most from softening, and that it is likely that the ideal treatment in most eyes will involve some combination of softening and stiffening to achieve a desired biomechanical result. In that regard, tissue stiffening may be rendered much more effective when combined with tissue softening to properly route forces and deformations to minimize damage.

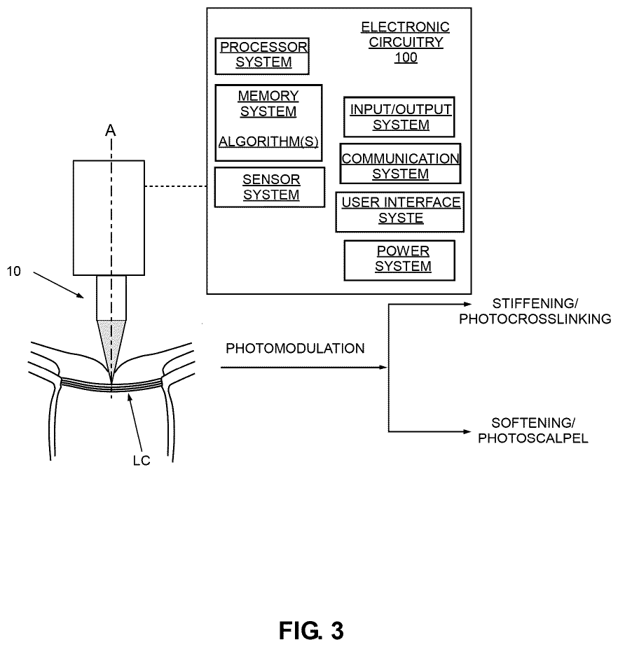

There are currently no methods available for treatment of, for example, glaucoma, which proceed through tissue softening or proceed through both softening and stiffening. By providing for both softening and stiffening, the results achieved could involve not just the modification, but potentially the ability to reverse adverse effects. Methods that can only stiffen tissue lack the ability to reverse course. Thus, improved or optimal effects may be achievable via a combination of both tissue stiffening and softening. In a number of embodiments hereof, both such effects can be obtained using two lasers or, alternatively, using a single laser as, for example, illustrated schematically in FIG. 3A. Laser 10 may be under software or robotic control (for example, under imaging guidance) as known in the surgical arts using electronic circuitry 100 including, for example, a processor system in communication with a memory system in which software algorithms are executable by the processor system to effect control and, in some cases, analysis. Imaging data and/or other sensor data (collected via a sensor system) can be used to guide the process, as well as to effect feedback control of certain aspects of the process. As known in the computer arts, electronic circuitry may, for example, include an input/output system, a communication system (which may provide for wired and/or wireless communication), a user interface system, and a power system.

In a number of studied embodiments, a control system/electronic circuitry 100 such as that illustrated in FIG. 3A was coupled to a Ti-Sapphire laser with a temporal pulse width of 140 fs and an 80 MHz repetition rate at a wavelength of 800 nm (that is, near-infrared irradiation). In a number of embodiments, the wavelength may, for example, be in the range of 600 to 1100 nm. A 25× NA 1.05 objective lens was used to focus the femtosecond laser. In the context of photomodulation, a single “application” or “dose” may be defined as laser pulses with a determined duration or dwell time (for example, a dwell time of 200 μs). The pulses may, for example, be scanned in a linear, planar, or any geometrical pattern. The application may be accompanied, for example, by an average laser power after the objective lens of 120 mW (equivalent to 1.5 nJ). A femtosecond oscillator (determining pulse width and repetition rate), pulse width, and wavelength may be readily determined for a specific application. In a number of embodiments, the pulse energy of the femtosecond laser after objective output is in the range from approximately 0.1 nJ to approximately 10 nJ, with a significant dependence on the numerical aperture (NA) of the objective. A lower NA objective necessitates higher energy. Photocrosslinking may, for example, be induced by a dosage of 5 to 45 units of photomodulation, while photoscalpel activation may, for example, occur with a dosage ranging from 30 to 100 units of photomodulation (depending upon pulse energy). The dwell time is inversely proportional to the required photomodulation dose. Dosage and dwell time are readily determined by one skilled in the art for a particular application and to achieve a desired result.

In a number of embodiments, a femtosecond laser using, for example, near-infrared femtosecond laser irradiation may be used to photoinduce (that is, induce via photons) either softening or stiffening of a one or more volumes of tissue without thermoacoustic damage or shock waves. Softening or stiffening may be induced via a single laser (for example, femtosecond laser) by controlling the cumulative or total time of application of the laser energy to effect changes in tissue (for example, collagen) microstructure. For example, multiple applications/treatments at a given energy level and duration may be applied and the number of such applications or treatments controlled. Less cumulative time of laser energy application may be used to induce stiffening. Such stiffening may, for example, occur via photon-induced crosslinking. Photon-induced crosslinking in, for example, collagenous tissue has been demonstrated but the underlying mechanism is not fully understood. Greater cumulative time of laser energy application results in tissue degeneration (for example, cutting) and softening (that is, increased compliance) of the tissue.

In the treatment of glaucoma, photoinduced modulation of biomechanical properties occurs in the tissues of the eye. For example, the photomodulation can occur in the collagenons tissue of the eye (for example, collagenous tissues of the back of the eye, including at least one of the sclera and lamina cribrosa). Of the tissues at the back of the eye, the LC is a particularly desirable target of such photomodulation of biomechanical properties. The LC is close to the axons and has less pigment than the sclera, facilitating photomodulation. Other tissue of the eye that may be targeted using the devices, systems, and methods hereof in the treatment of glaucoma include collagenous tissue of at least one of the trabecular meshwork, Schlemm's canal, the episcleral. In that regard, the devices, systems, and methods hereof may be used to modulate biomechanical properties of tissue to alter IOP by affecting aqueous outflow from the eye. The axial (see axis A in FIG. 2) and lateral resolutions of lasers such as femtosecond lasers are sufficient to, for example, limit, minimize or eliminate photoinduced damage to tissue not targeted in treatments including, for example, photoinduced damage to blood vessels and axons.

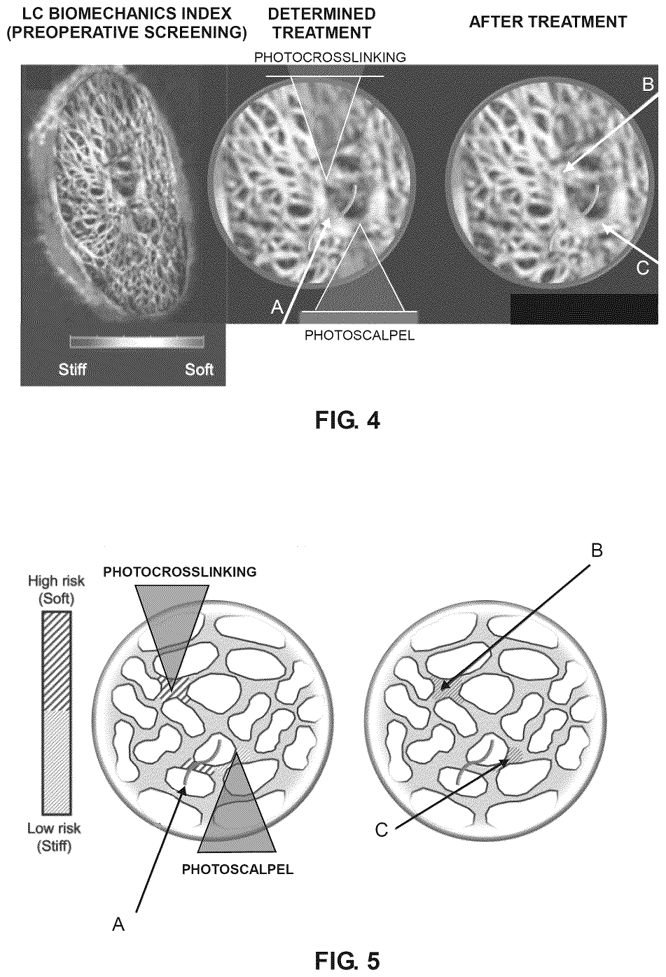

FIGS. 4 and 5 illustrates representative examples of a process hereof including preoperative screening of eye tissue such as the LC in which an LC biomechanics index is created. Methods of underlying determination of biomechanical properties of tissues of the eye are, for example, described in Vorhees, A. P., et al., So-Called Lamina Cribrosa Defects May Mitigate TOP-Induced Neural Tissue Insult, Investigative Ophthalmology & Visual Science, Vol. 61, 15 (2020); Sigal I. A., et al., IOP-Induced Lamina Cribrosa Displacement and Scleral Canal Expansion: An Analysis of Factor Interactions Using Parameterized Eye-Specific Models, Investigative Ophthalmology & Visual Science, 52(3): 1896-907 (2011); and Sigal, I. A., IOP-Induced Lamina Cribrosa Deformation and Scleral Canal Expansion: Independent or Related?. Investigative Ophthalmology & Visual Science, 52(12): 9023-32 (2011), the disclosures of which are incorporated herein by reference. FIG. 4 was prepared using structured polarized light microscopy (SPLM). See, for example, Yang, B., et al., Structured polarized light microscopy for collagen fiber structure and orientation quantification in thick ocular tissues. Journal of biomedical optics, 23(10), 1-10(2018). The preoperative screening provides the information required to determine one or more volumes of tissue to be stiffened via photocrosslinking and to determine one or more different volumes of tissue to be softened via photodegradation/photoscalpel. In the representative example of FIGS. 4 and 5, the preoperative screening reveals that a soft region in the LC exhibited significant deformation, posing a high risk, Photocrosslinking of one or more volumes of tissue in that region may be used to effectively stiffen that region, thereby protecting the surrounding tissue. The preoperative screening further indicates that another soft region of tissue contains blood vessels (arrow A indicates a vessel inside a beam), raising concerns about damage from photomodulation in that region. In FIGS. 4 and 5, arrow B in indicates the area in which preoperative screening revealed that a soft region exhibited significant deformation, posing a high risk. Photocrosslinking is used to effectively stiffen that region, protecting the surrounding neural tissue. Arrow C indicates the area in which preoperative screening revealed that a soft region contains blood vessels, raising concerns about potential irreversible damage from photomodulation in that area. Rather than stiffening the area designated by arrow C via photocrosslinking, the use of photoscalpel can soften adjacent stiff regions, allowing them to share more of the burden and alleviating the load from the overloaded beam to their left (in the orientation of the figure).

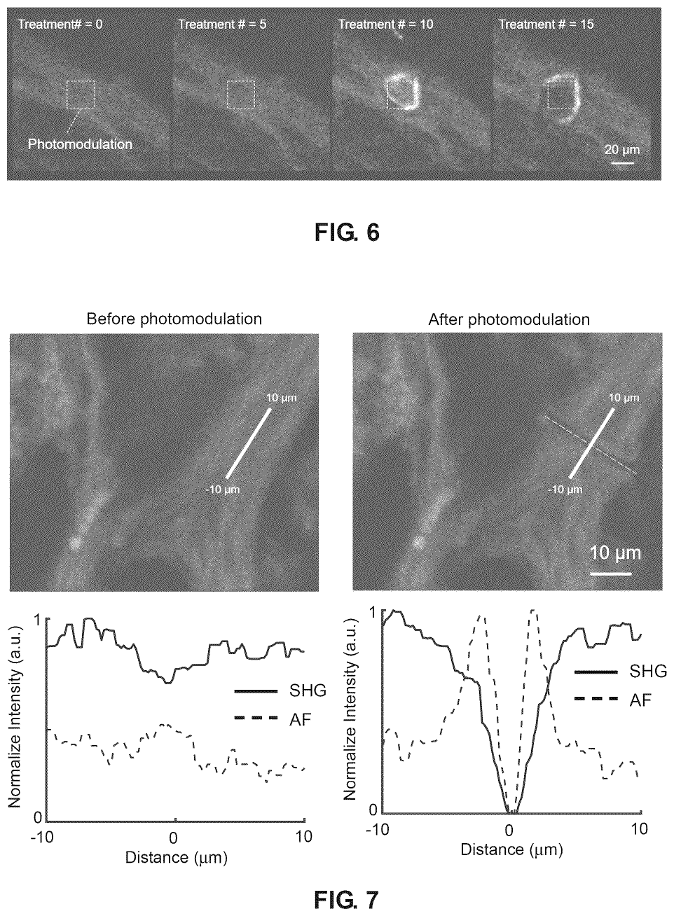

In characterizing the effect of photomodulation, changes in SHG and TPAF signals (as, for example, measured by a multiphoton microscope system coupled to the laser) accompanying an increase in the number of photomodulation treatments revealed that collagen involves nonlinear photomodulation responses with different stages under near-infrared femtosecond laser irradiation. As illustrated in FIG. 6, within 5 treatments, SHG and TPAF signals had no remarkable change. After 10 treatments, only the TPAF signal increased significantly in the treatment area and along a part of the outer boundary of the treatment area. The increased TPAF signal was associated with the formation of new photoproducts and enhanced collagen crosslinks, which is referred to as photocrosslinking herein. After 15 treatments, the decrease in SHG and TPAF signals in the treatment area indicates laser-induced collagen degradation, and associated structural changes within the collagen, referred to as photoscalpel herein. The SHG and TPAF signals were still observed along the outer boundary of the cavity.

In a number of studies, linear photomodulation treatments were applied on a lamina beam. A decreased signal in SHG indicates photoscalpel, whereas an increased fluorescence signal on the cutting edge indicates photocrosslinking. The profiles of SHG and TPAF signals in the region of interest show the lateral resolutions of photoscalpel and photocrosslinking were about 3 μm and 6 μm, respectively (FIG. 7). The theoretical imaging resolution is about 0.4 μm.

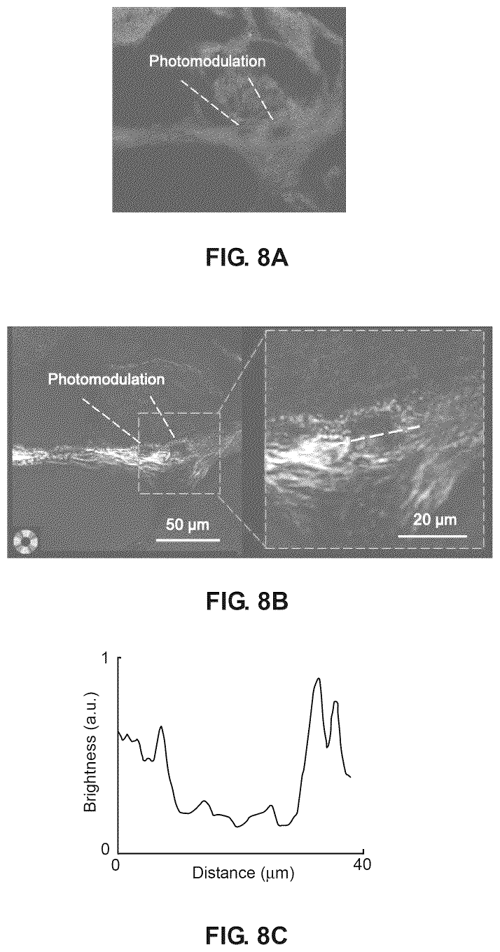

IPOL imaging showed that photomodulation did not cause thermoacoustic damage or shock waves and changes in collagen fiber orientation in the outer boundary of the treatment area (FIG. 8A). The IPOL image shows that photoscalpel did not change the collagen organization around the border of the cavity (see FIG. 8B). The photoscalpel regions appears darker in the treatment area than the surrounding region, indicating that the treatment regions is thinner. The decrease in the brightness of the IPOL image under femtosecond laser irradiation revealed that the thickness of collagen beams decreased to about half, (that is, by 8 μm; see FIG. 8C) since the brightness of the IPOL image is proportional to the retardance. See Yang, B., Lee, P. Y., Hua, Y., Brazile, B., Waxman, S., Ji, F., Zhu, Z., Sigal, I. A., 2021. Instant polarized light microscopy for imaging collagen microarchitecture and dynamics. Journal of Biophotonics 14, e202000326 (2021). This indicates that the axial resolution of photomodulation in the study was better than 8 μm. Resolutions may be improved through further optimization using know engineering principles.

No bubbles were found in the IPOL image, and the orientation of the cutting edge was consistent with the surrounding. Such observations provided further evidence that laser induced chemical reactions rather than thermal or thermoacoustic effect are responsible for changes induced in the collagen.

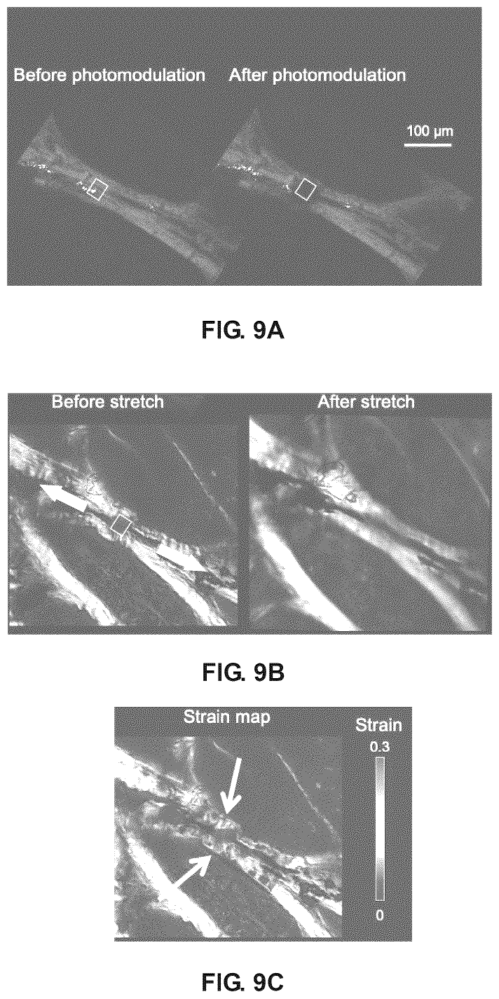

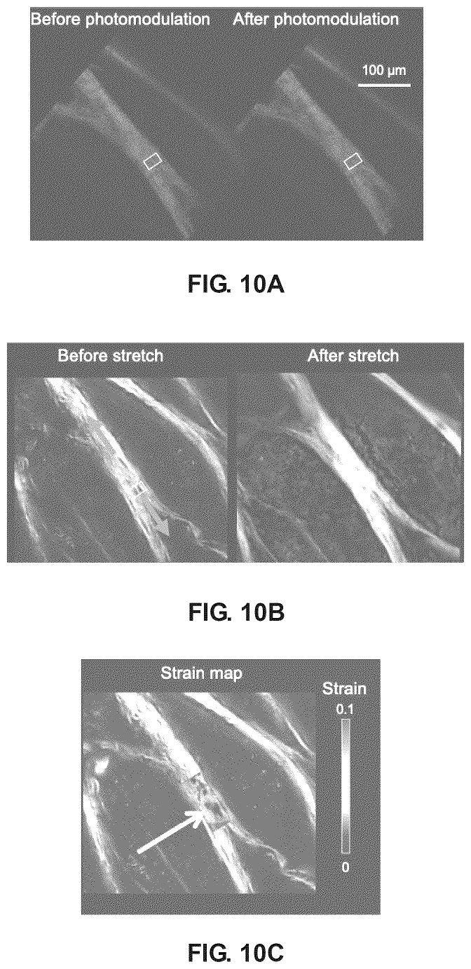

Strains of laminar beams with and without photomodulation treatments were measured and compared using stretch testing (FIGS. 9A through 10C) using a biaxial stretching device compatible with the microscope as discussed below. The mechanical properties of lamina beams after photomodulation were thereby studied. The treatment region was located by comparing SGH and IPOL images. The tissue section was stretched and imaged in a step-by-step process. Since a lamina beam is a linear and relatively uniform structure, the deformation is relatively simple. The deformation during stretch was tracked and calculated. The change in mechanical properties was identified by the strain difference between treated and untreated regions. When the treated region has a lower strain, it indicates a stiffer region. If the treatment region has a higher strain, it indicates a softer region.

In the first case, the occurrence of photoscalpel (collagen loss) was identified from the loss of SHG signals on the lamina cribrosa beam (FIG. 9A). There are two bending collagen bundles located at the treated region (FIG. 9B). The collagen bending region shows remarkably high strain under biaxial stretch testing compared to neighboring regions (FIG. 9C). This observation indicates that the region with collagen bending was less efficient at carrying loads. In the other case, there was no significant change in SHG and collagen loss after photomodulation, indicating that the femtosecond laser irradiation was insufficient to induce photoscalpel (FIG. 10A and FIG. 10B). A lower strain was found in the photomodulation region, indicating an increase in tissue stiffness (FIG. 10C). A lower strain supports the determination that photocrosslinking occurred in the photomodulation region, even though there was also no significant change in TPAF signals after photomodulation.

In the studies hereof, it was demonstrated that near-infrared femtosecond laser irradiation allowed controllably changing the mechanical properties of collagen tissues without the use of photosensitizers. The precision of photomodulation was at the micrometer level in both lateral and axial directions and can be improved through further optimization. In IPOL images, it was found that no change in collagen architecture in the outer boundary of the treatment area indicates that photomodulation was based on laser-induced low-density plasma (which may be viewed simply as free electrons) without the production of thermoacoustic damage and shock waves. Quantifying of induced changes in the mechanical properties of collagen tissues using femtosecond laser irradiation may be characterized. The approach hereof may, for example, be used in therapeutic applications such as in intraocular surgery, cartilage reshaping, and tissue engineering. Techniques hereof may, for example, also have potential to contribute to treatment of biomechanics-related pathologies of the eye other than glaucoma, such as myopia.

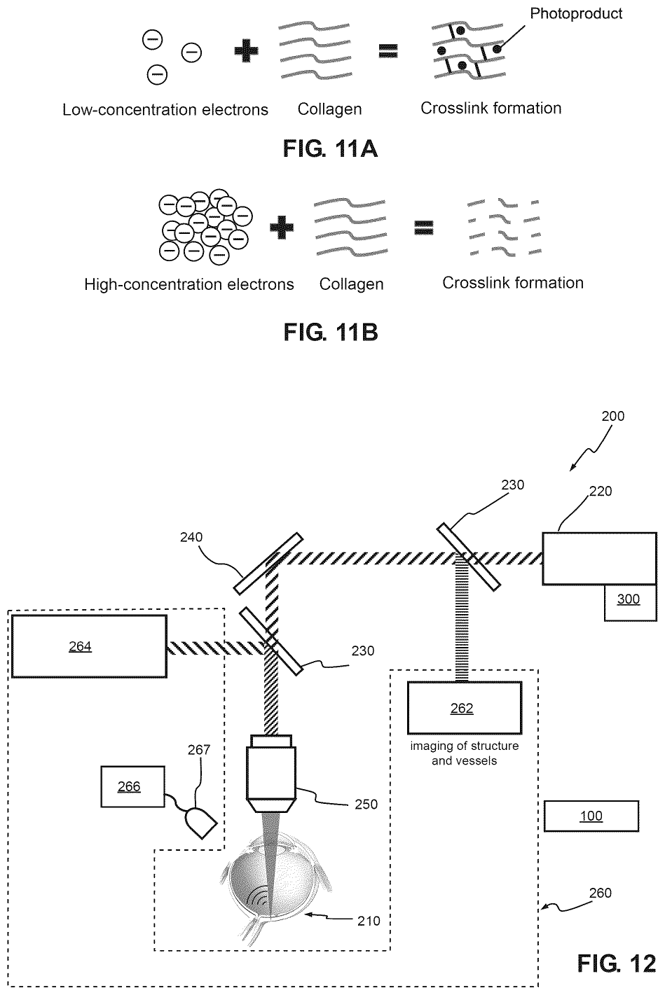

As described above, photomodulation may be categorized into two photochemical reactions: collagen crosslinking (photocrosslinking) and collagen degradation (photoscalpel). One again, it was found that the dominant chemical reaction was related to the number of treatments. Without limitation to any mechanism, the series of photomodulation treatments suggests a photomodulation theory wherein a low-concertation reactant (that is, free electrons) forms collagen crosslinks (photocrosslinking), whereas a high-concertation reactant induces collagen degradation (photoscalpel) (see FIGS. 11A and 11B). Free electrons can participate in collagen crosslinking processes, influencing the mechanical properties of collagen fibers. Meanwhile, free electrons can also contribute to collagen degradation as a result of the generation of reactive oxygen species. The concentration of reactants could significantly impact the rate and outcome of chemical reactions. Therefore, balancing those processes is important for controlling photomodulation effects. The theory is also supported by the signal on the outer boundary of the treatment area. First, the reactant can diffuse, and thus the changed signal is not just located in the treated area. The outer boundary of the treatment area also exhibited an increased TPAF signal. Second, the concertation of the diffused reactant in the outer boundary is less than the one in the treated area. The dominant chemical reaction is photocrosslinking on the outer boundary due to the low-concertation reactant. In the resolution study discussed above, it was found that the photomodulation resolution is significantly worse than (˜10×) theoretical imaging resolution. That observation is another indication of chemical reactant diffusion. The concept of the concentration of free electrons in the photomodulation theory discussed above differs from the density of plasma in femtosecond laser effects. The former pertains to the equilibrium of chemical reactions, while the latter is relevant to nonlinear optical effects.

Once again, representative potential applications of photomodulation as described herein include prevention and treatment of glaucoma by modulating the stiffness of the lamina cribrosa. Biomechanically, reducing the deformations of lamina cribrosa within the optic nerve head may, for example, increase the resistance to glaucomatous mechanical insult. The lamina cribrosa is where the retinal ganglion cell axons exit the globe. It is at the lamina cribrosa that the mechanical insult to axons is thought to be worst, and therefore the location where the devices, systems, and methods hereof may have the most effective in protecting the delicate neural tissues. The rational and direct modulation of the local mechanical properties of the lamina cribrosa through a combination of softening and stiffening can reduce the mechanical insult to the neural tissues and protect the retinal ganglion cell axons and their function. Such a therapy would be independent of, and synergistic with, current therapies based on lowering pressure. Importantly, this therapy could also be directed to patients suffering neural tissue damage at normal pressure. Accessing the posterior pole of the eye using laser may, for example, occur through the cornea. Further studies will improve the understanding of photomodulation effects on neural tissues and vascular networks. As described above, when modifying collagen architecture to release the mechanical insult, it is desirable to avoid directly damaging vessels as well as any resulting changes in mechanical support to vessels should be considered.

In, for example, performing a biomechanical analysis of the tissue of the eye (for example, in evaluating LC biomechanics) a representative methodology includes compliance testing, digital image correlation (DIC) and biomechanical analysis. DIC is an approach that enables the assessment of the deformation and motion of objects, which is achieved by comparing digital images of the region of interest before and after deformation. In that regard, one may image the tissue at a first pressure, alter the pressure to a second, higher pressure, then image the tissue once again at the second, higher pressure to determine deformation and motion. Precise pressure changes may be achieved in animals subjects using cannulas into the eye. For human patients, one may gently push on the eye ((for example, by softly push on the sclera) to increase pressure. Such pushing causes no discomfort to the patients and has been shown safe. Tools have been developed to push into anterior sclera to increase eye pressure without distorting the cornea. It may also be possible to use the natural or intrinsic changes in pressure resulting from deformations induced by the heartbeat and, therefore, not require any extrinsically induced deformation to the eye to change pressure. See, for example, Kim, J., et al. Strain by virtual extensometers and video-imaging optical coherence tomography as a repeatable metric for IOP-Induced optic nerve head deformations. Experimental eye research, 211, 108724(2021). Deformation of tissues in other regions of interest for application of DIC may be induced using various techniques as known in the art.

The images created at different pressures are analyzed by DIC to reveal tissue movements and deformations resulting from the pressure change. The movements and deformation are, in turn, analyzed using biomechanics. With a database including data of a sufficient number of eyes, one may, for example, develop correlations between characteristics of the eye and biomechanical indices (that is, associated regions with a certain characteristic image with a higher/lower biomechanical index). Artificial intelligence may be used in creating algorithms and models to determine such correlations (for example, machine learning, image recognition, etc.) as known in the computer arts.

As described above, a potential concern when doing photomodulation of eye tissue is that it could damage the vasculature, which could result in hemorrhaging and potentially later cause problems with perfusion and oxygenation. It has previously been suggested that every LC beam includes a capillary within. If that were the case, it would make it difficult to achieve photomodulation of LC beams without affecting the capillary/blood vessels. As described above, it has been shown that it is not the case that every LC beam includes a vessel. While many LC beams do include blood vessels, not all do. Of those that include vessels, the vessel is sometimes only partially within the beam, or to a side of the beam, leaving open the potential for photomodulation thereof. Vessels may also be positioned in different locations relative to LC beams and pores (for example, outside of a beam, crossing a pore, or crossing a beam). While photomodulation of some LC beams must be done carefully to avoid damage, the distribution of vessels in the LC provide the opportunity for photomodulation thereof as without significant difficulty. One may, for example, photomodulate an LC beam in which there is no vessel present. Further, in the case of an LC beam including a vessel, one may photomodulate a portion or portions of the LC beam through which the vessel does not pass.

To avoid damage to the vasculature in the ONH (or another region of interest in which the potential for damage to vasculature exists), it is highly desirable to visualize and characterize the vascular network in the ONH (or another region of interest) before performing photomodulation. In such a characterization process, is not necessary to fully reconstruct the vasculature. It is typically desirable or necessary to characterize only those regions or volumes in which one intends to perform photomodulation. In the case of photomodulation via laser energy, such photomodulation can only be achieved in target tissues in which there is a line of sight or unobstructed view to the target tissue. Although a technique such as OCT angiography or OCTA, which uses motion contrast to detect blood flow from OCT data, is limited in depth, it is well suited for characterization of the vasculature in those regions of the eye in which photomodulation is possible. Other imaging techniques such as ultrasound imaging, fluorescence etc. may be used.

FIG. 12 illustrates schematically an embodiment of a system 200 hereof that can be used in altering the biomechanics of collagen (that is, at least one of softening and stiffening) in, for example, eye 210 of FIG. 12. In the illustrated embodiment, a laser system 220 may, for example, include one or more lasers such as femtosecond lasers. Various optical elements such as beamsplitters 230 may be used for separating, splitting, or combining beams of light. One or more mirrors such as scan mirror 230 may be used to reflect/redirect light. Optical elements such as one or more objective lens 250 as known in the optics and imaging arts may be positioned between the tissue of the region of interest (eye 210 in the illustrated embodiment) and other system components for focusing light energy. System 200 further includes an imaging system 260 (encompassed by broken lines in FIG. 12) including one or more imaging devices or systems to image components such as tissue structure, vasculature, photomodulation side products etc. In the illustrated embodiment, an OCT/OCTA system 262 may be provided to image tissue structure and vasculature. A photomultiplier tube/multiphoton microscope system 264 may be provided to enable, for example, SHG/multiphoton fluorescence imaging of collagen, imaging of vasculature, imaging of neural components, and imaging of photomodulation side products. A ultrasound imaging system 266 including an ultrasonic transducer 267 may be provided for photoacoustic imaging of vasculature. One or more robotic systems 300 (for example, including one or more robotic arms as known in the robotic surgical arts) may be provide to, for example, control application of laser energy from laser system 200. Electronic circuitry 100 as described above, which may be centralized or distributed over multiply components to varying degrees, may be provided to, for example, perform one or more of controlling one or more components of system 200, acquisition of data, analysis of data, etc.

Tissues (and, particularly collagenous tissue) other than tissue in the eye may also benefit from the devices, systems, and methods hereof. Lasers have been used for many years to remove and ablate tissues, in a coarse version of the photoablation. The ability to alter tissue locally in a precise and controlled fashion opens possibilities of more refined and nuanced effects. There are many areas of treatment that rely on changes of tissue biomechanical properties. Such treatments focus on either stiffening or softening, but not both. Many of such techniques use chemicals, which are far less precise and more invasive than photomodulation. Many such treatments may benefit from a nuanced, rational approach of using a determined pattern of softening and stiffening to achieve desirable biomechanical properties. For example, in vascular mechanics, a number of studies have investigated collagen cross-linking as a method to strengthen blood vessels and arterial walls. Photomodulation could, for example, be used to stop a positive-feedback loop in which the tissues of the vessel wall stiffen, altering the regional mechanics and flow, leading to further stiffening. The process creates an aneurysm that eventually breaks with extremely high mortality. Therapies such as ultrasound therapy have been shown to reduce stiffness in muscles and joints while increasing the range of motion. In dermatology, collagen cross-linking has been studied as a potential treatment for skin conditions. The ability to precisely tailor mechanical properties via a predetermined pattern of softening and stiffening may allow for better outcomes (for example, softer scar tissues). In orthopedics, collagen cross-linking has been investigated for its potential to enhance the mechanical properties of tendons and ligaments. In dentistry, collagen cross-linking has been explored in, for example, stabilizing dentin collagen.

### Representative Experimental Methods and Systems

Photomodulation. A commercial multiphoton microscope system (FV1000MPE, Olympus, Tokyo, Japan) was used for photomodulation and for multiphoton imaging used to localize and evaluate the effects on the collagen. The system was coupled to a Ti-Sapphire laser (Chameleon Vision II, Coherent, Santa Clara, USA) with a temporal pulse width of 140 femtosecond (fs) and an 80 MHz repetition rate at a wavelength of 800 nm. A 25×NA 1.05 water-immerse objective lens (Olympus, Tokyo, Japan) was used to focus the femtosecond laser. For photomodulation, “treatment” consisted of laser pulses (200 μs dwell time) covering a linear or a planar treatment area, with an average laser power after the objective lens of 120 mW. For multiphoton imaging, detection bandwidths of second harmonic generation (SHG) and two-photon autofluorescence (TPAF) signals were 350-450 nm and 500-550 nm, respectively, with an average laser power after the objective lens of S mW. The laser can generate second harmonic generation to visualize collagen and two-photon autofluorescence signals at the same time, and two detection bandwidths may be used for the two signals.

Instant Polarized Light Microscopy. Instant polarized light microscopy (IPOL) was used to further examine the collagen microstructure and orientation after photomodulation. See Yang, B., Lee, P. Y., Hua, Y., Brazile, B., Waxman, S., Ji, F., Zhu, Z., Sigal, I. A., 2021. Instant polarized light microscopy for imaging collagen microarchitecture and dynamics. Journal of Biophotonics 14, e202000326 (2021). IPOL was implemented with a commercial inverted microscope (IX83; Olympus, Tokyo, Japan), a color camera (DP74, Olympus, Tokyo, Japan), and a 10× strain-free objective (numerical aperture 0.13). IPOL allows label-free visualization and quantification of collagen structure and orientation via color information for each pixel. Briefly, a region with low or no birefringence appeared dark since the polarizations of the white light had no change and thus was blocked. A birefringent sample, such as collagen, appeared colorful since IPOL differentiated the polarizations with wavelengths and thus changed the spectrum of the white light. The colorful light was acquired by a color camera to produce true-color images indicating collagen fiber orientation and structure. Once can view the colorful collagen directly through the eyepiece or use a color camera to record the results. The true color of the image is related to the collagen fiber orientation and a dark background is present because of less collagen in the background. The imaging speed was almost limited by the camera speed. The methodology is thus also suitable for mechanical testing.

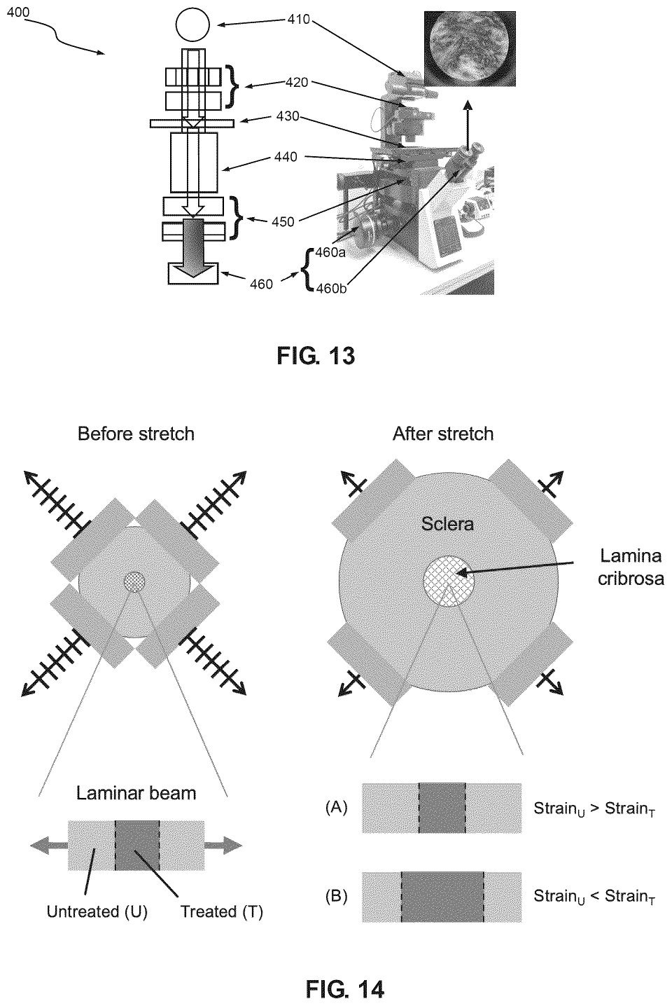

A representative embodiment of an IPOL system 400 is illustrated schematically and via a photograph in FIG. 13. The illustrated IPOL system includes a light source 410 (for example, a source of white light), a polarization encoder 420, a sample stage 430 to support a sample, an objective lens 440, a polarization decoder 450 and an image presentation/display system 460 (including, for example, a camera 460a and an eyepiece 460b).

Sample Preparation. Sheep eyes, approximately one year old, were acquired from a local abattoir and processed within four hours after death to obtain coronal sections of unfixed tissues. Briefly, the muscles, fat, and episcleral tissues were carefully removed. The optic nerve head or cornea regions were isolated using an 11-mm-diameter trephine and embedded in optimum cutting temperature compounds (Tissue-Plus; Fisher Healthcare, TX, USA). Samples were then snap-frozen in liquid nitrogen-cooled isopentane and sectioned coronally at a thickness of 16 μm,

Biaxial Stretch Testing. Biaxial stretch testing was used to evaluate the difference in mechanical behavior between tissue regions with and without photomodulation. First, each fresh tissue section of the optic never head was placed on the microscope slide without coverslips for photomodulation. After photomodulation, the section was pre-imaged with IPOL. Pre-imaging was used to localize the regions with photomodulation by comparing multiphoton and IPOL images. Due to the limited field of view of the objective, the image of the whole section was acquired using mosaicking. Images with 20% overlap were acquired using a translational stage and stitched into mosaics using Fiji. After pre-imaging, the tissue section was then mounted on a custom biaxial micro-stretcher system following the previously reported methodology. Lee, P.-Y., Yang, B., Hua, Y., Waxman, S., Zhu, Z., Ji, F., Sigal, I. A., 2022b. Real-time imaging of optic nerve head collagen microstructure and biomechanics using instant polarized light microscopy. Experimental Eye Research 217, 108967 (2022). Briefly, two size-fit pieces of silicone sheeting (Medical Grade, 0.005″; BioPlexus, AZ, USA) were used to sandwich the tissue section to prevent curling or tears at the clamp points. The section was simultaneously stretched equally along two orthogonal axes in 0.5 mm stretch steps and imaged with IPOL quasi-statically. At each stretch step, multiple images were captured to cover the entire laminar region in a mosaic. Autofocus was applied to avoid out-of-focus images during the stretch. Since the treatment regions had been identified in the pre-imaging session, we can easily localize these regions during the stretch.

Strain Calculation. Strain calculation was used to quantify the tissue deformation under biaxial stretch testing. A custom MATLAB program was developed for manually placing rotatable rectangular regions on laminar beams with photomodulation. Each rectangular region created seed points. These seed points were tracked using digital image correlation at each step. See Zhong, F., Quan, C., Efficient digital image correlation using gradient orientation. Optics & Laser Technology 106, 417-426 (2018) and Zhong, F., Wang, B., Wei, J., Hua, Y., Wang, B., Reynaud, J., Fortune, B., Sigal, I. A., A high-accuracy and high-efficiency digital volume correlation method to characterize in-vivo optic nerve head biomechanics from optical coherence tomography. Acta Biomaterialia 143, 72-86 (2022). The MATLAB program allowed manual adjustment for the tracking results when the tracking results were inaccurate. The strains were calculated using the Savitzky-Golay filter-based method and then the first principal strain was extracted from the Green-Lagrange strain tensor, as a measure of the largest stretch. The difference in strain between the treated and untreated regions was used to identify if the treated region became softer or stiffer (FIG. 14).

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A method of modifying biomechanical properties of tissue in a region of interest comprising:
application of laser energy to one or more first volumes of tissue in the region of interest to cause softening via photodegradation; and
application of laser energy to one or more second volumes of tissue within the region of interest to cause stiffening via photocrosslinking, the one or more second volumes are different from the one or more first volume.

2. The method of claim 1 further comprising determining modifications of the biomechanical properties of the tissue in the region of interest and determining the one or more first volumes of tissue and the one or more second volumes of tissue to achieve the determined modifications prior to application of laser energy to the one or more first volumes of tissue and application of laser energy to one or more second volumes of tissue.

3. The method of claim 2 further comprising creating a biomechanical index or map of the region of interest before application of laser energy to the one or more first volumes of tissue and before application of laser energy to the one or more second volumes of tissue.

4. The method of claim 1 wherein the one or more first volumes of tissue comprises collagen and the one or more second volumes of tissue comprises collagen.

5. The method of claim 4 wherein a first laser is used to apply laser energy to the one or more first volumes of tissue and a second laser is used to apply laser energy to the one or more second volumes of tissue.

6. The method of claim 4 wherein a single laser is used to apply laser energy to the one or more first volumes of tissue and to the one or more second volumes of the tissue.

7. The method of claim 1 wherein the biomechanical properties of the tissue are modified through thermoacoustic effects or through photochemical effects.

8. The method of claim 1 wherein the biomechanical properties of the tissue are modified in the absence of thermoacoustic effects.

9. The method of claim 1 wherein a femtosecond laser is used for at least one of application of laser energy to the one or more first volumes of tissue and for application of laser energy to the one or more second volumes of tissue.

10. The method of claim 1 wherein a femtosecond laser is used for application of laser energy to the one or more first volumes of tissue and for application of laser energy to the one or more second volumes tissue.

11. The method of claim 10 wherein the total time of application of laser energy to each of the one or more first volumes of tissue is less that the total time of application of laser energy to each of the one or more second volumes of tissue.

12. The method of claim 11 wherein each of the one or more first volumes of tissue and each of the one or more second volumes of tissue is tissue of the eye.

13. The method of claim 12 wherein each of the one or more first volumes of tissue and each of the one or more second volumes of tissue is tissue of the trabecular meshwork, Schlemm's canal, the episclera, the sclera or tissue of the lamina cribrosa.

14. The method of claim 12 wherein each of the one or more first volumes of tissue and each of the one or more second volumes of tissue is tissue of the lamina cribrosa.

15. The method of claim 11 wherein each of the one or more first volumes of tissue and each of the one or more second volumes of tissue are located on one or more beams of the lamina cribrosa which do not include a blood vessel.

16. The method of IS wherein the biomechanical properties of the tissue are modified to treat glaucoma or to prevent glaucoma.

17. The method of claim 10 wherein the laser energy applied to the one or more first volumes of tissue and the laser energy applied to the one or more second volumes of tissue has an axial resolution of not more than 40 μm, not more than 30 μm, not more than 20 μm, or not more than 10 μm.

18. The method of claim 10 wherein the laser energy applied to the one or more first volumes of tissue and the laser energy applied to the one or more second volumes of tissue has a lateral resolution of not more than 40 μm, not more than 30 μm, not more than 20 μm, or not more than 10 μm.

19. The method of claim 3 wherein the region of interest is tissue of the eye and creating the biomechanic index comprising comparison of images created at different pressures within the eye, wherein the different pressures are created either intrinsically or extrinsically.

20. A method of treating or preventing glaucoma comprising softening tissue in a region of interest in the eye.

21. The method of claim 20 wherein the collagenous tissue of the eye is collagenous tissue of at least one of the trabecular meshwork, Schlemm's canal, the episclera, the sclera and the lamina cribrosa.

22. The method of claim 20 wherein the collagenous tissue of the eye is collagenous tissue of the lamina cribrosa.

23. The method of claim 20 wherein the tissue in the region is softened via the application of laser energy.

24. The method of any claim 20 further comprising applying the laser energy to one or more second volumes of collagenous tissue of the eye to cause stiffening in the region of interest, the one or more second volumes being different from the one or more first volumes.
